(19) United States
(12) Patent Application Publication
(10) Pub. No.: US 2002/0099310 A1
(43) Pub. Date: Jul. 25, 2002

(54) Gastrointestinal-tract sensor

(75) Inventors: Yoav Kimchy, Haifa (IL); Yona Bouskila, Yokneam (IL); Roni Amrani, Yokneam (IL); Udi Antebi, Kiryat Bialik (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(73) Assignee: V-Target Ltd.

(21) Appl. No.: 09/765,316

(22) Filed: Jan. 22, 2001

(51) Int. Cl. A61B 5/103; A61B 5/117

(52) U.S. Cl. 600/587; 600/593

(57) ABSTRACT

A method for detecting a site of interest in a gastrointestinal tract, wherein the method includes sensing a length traveled by a sensor in a gastrointestinal tract from a reference point to a site of interest in the gastrointestinal tract.

### FIELD OF THE INVENTION

The present invention is generally in the field of the diagnosis of ailments such as cancer, and relates to a method and system for tracking and communicating wit a diagnostic instrument that travels in, and collects data from, the gastrointestinal tract.

### BACKGROUND OF THE INVENTION

There is a large occurrence of cancer in the digestive tract of people above the age of 50, such as in the form of cancer in the stomach, small intestine (duodenum, jejunum and ileum) or large intestine (cecum, colon and rectum). Detection of such cancer in the early stage can markedly increase the chances of survival.

Disorders to the small intestine and the colon also present significant health risks. Such disorders include, for example, irritable bowel syndrome, fluxional diarrhea, ulcerative colitis, collagenous colitis, microscopic colitis, lymphocytic colitis, inflammatory bowel disease, Crohn's disease, infectious diarrhea, ulcerative bowel disease, lactase deficiency, infectious diarrhea, amebiasis, and giardiasis, for example.

Ulcerative colitis is an inflammatory disease of the colon characterized by chronic diarrhea which is often bloody. Ulcerative colitis may affect only a portion of the colon or it may affect the entire length of the colon, in which case the disease is designated pan-ulcerative colitis.

Collagenous colitis is a condition characterized by chronic diarrhea and abnormalities of the colonic mucosa.

Crohn's disease, also referred to as regional enteritis, is characterized by inflammation, thickening and ulceration of any of various parts of the intestine, especially the ileum.

Giardiasis is an example of an infectious disease characterized by diarrhea, which is often chronic and which is caused by a parasite (the protozoan Giardia lamblia). Other parasites and infectious agents, such as bacteria and viruses, cause diarrhea which may be acute or chronic.

To date, there is no satisfactory method for screening large populations for these kinds of diseases at an early stage. Endoscopy of the digestive tract is performed only when patients become symptomatic. However, at such a symptomatic stage, the cancer may have already been well developed with metastasis sent to other parts of the body. This state of affairs leaves much to be desired, with generally poor prognosis and low survival statistics for the patients.

An example of endoscopy is described in U.S. Pat. No. 5,984,860 to Shan. In this document, a pass-through duodenal enteroscopic device utilizes the natural contraction wave of the small intestine to propel the device through the small intestine at about the same speed as any other object therein. The exterior of the device is streamlined over the greater portion thereof with a video camera and illumination source at the forward end of the device. Covering the camera lens and illumination source is a transparent inflatable balloon adapted to gently expand the small intestine immediately forward the camera for better viewing. A small diameter communication and power cable is wound within the device and unwinds through an aperture in the rear of the device as the device moves through the small intestine. Upon completion of movement through the small intestine the cable is automatically separated from the device permitting the cable to be withdrawn through the stomach and intestine. The device continues through the large intestine and passes from the patient through the rectum.

Endoscopic technology has long since matured to visualize the entire colonic mucosa via colonoscopy and to visualize the esophagus, stomach and first 20 cm of the small intestine (duodenum). There is as of yet no fully satisfactory way of visualizing the full length of the small intestine. Currently, there are two types of endoscopes used to visualize the small intestine, the push endoscope and the Sonde (pull type) enteroscope. These two devices are very limited in their usefulness. The most efficacious way of inspecting the entire small bowel mucosa is to perform an intraoperative enteroscopy where the surgeon performs a laparotomy on the patient and actually moves the small intestine over the enteroscope. The enteroscope is driven by a gastroenterologist. Such a procedure is fully invasive and obviously an expensive and extreme measure to inspect the intestinal mucosa.

Visualization of the small intestine is important especially in patients who have occult gastrointestinal blood loss with no obvious source from the esophagus, stomach or colon. It is also important to examine the small intestine in patients with abdominal pain of unexplained origin and in patients with known diseases such as Crohn's disease or carcinoid syndrome. Barium small bowel follow-through is used most often to examine the small intestine, because of its ease and cost. However, this procedure rarely produces sufficient information for diagnoses. Physicians would usually prefer to visually inspect the small intestine mucosa if an adequate technique was available.

When a physician wishes to pursue investigation of the small intestine, the physician is left with the options of push enteroscopy, Sonde (pull type) enteroscopy or open intraoperative enteroscopy. Each of these technologies has failed to achieve widespread use because of the inherent drawbacks in each procedure.

The push enteroscope is similar in length and maneuverability to a colonoscope. However, trying to push a scope beyond the Ligament of Treitz is quite difficult because of the turns of the small bowel. At best most push enteroscopes can visualize less than half of the small intestine.

The Sonde enteroscope is a narrow device about 300 cm long. The Sonde device is inserted into the stomach and then is allowed to advance through the small intestine by peristalsis. Approximately six hours are required to pass this scope through the entire length of the small intestine because of the resistance to movement. The device allows visualization of the entire small intestine when successfully passed, however, the great length of time for the procedure is a definite drawback.

The intraoperative enteroscope procedure is done when a diagnosis of small bowel pathology is sought and less invasive tests have been non-diagnostic. The procedure requires a laparotomy by a surgeon in combination with small bowel enteroscopic viewing by a gastroenterologist working in concert to advance the scope over the entire length of the small intestine. It is obviously much more risky to the patient and involves high costs and intensive use of resources.

Another common method in the art, particularly used for screening for digestive tract cancer, is sampling blood in the stool. This method is not very sensitive, because blood is released when comparatively large polyps develop, and sometimes there is no release of blood to the stool until very late in the development of the disease.

Radiographic inspection of the gastrointestinal tract has long been used to examine the gastrointestinal tract for benign and cancerous growths. However, there is often little difference in X-radiation absorption between healthy gastrointestinal tract tissue and the aberrant growths sought to be identified. Fortunately, in gastrointestinal tract examinations, compounds of barium, an alkaline earth metal with a large nucleus and hence a high X-radiation stopping capacity, and gas releasing effervescent substances, which stop little radiation, can be introduced to produce large differences X-radiation absorptions. For example, in stomach examinations a patient typically swallows a combination of an effervescent substance and a barium compound. The barium compound is first received by the fundus of the stomach and then slowly released. As release occurs, the barium compound flows primarily along the troughs in the folds of the body of the stomach and is collected at the duodenal bulb. Hence some areas, such as the fundus and duodenal bulb have high levels of barium and exhibit high X-radiation absorptions while other areas, such as the ridges of the stomach folds, retain little barium and absorb low levels of X-radiation, while still other areas, such as the troughs in the body of the stomach, have intermediate levels of barium and exhibit intermediate X-radiation absorptions. In a well taken stomach radiograph, an almost three dimensional impression is created of the body of the stomach, allowing aberrant structure on the inner lining of the stomach to be seen as interruptions or diversions of the barium flow pattern.

Unfortunately, with radiographic elements that are currently available for gastrointestinal imaging it is generally difficult if not impossible to capture image detail in both the areas that are either primarily gas-filled, containing low levels of barium, or areas in which barium is collected or concentrated. The areas with low levels of barium often appear as physiological featureless black areas in the radiograph, since a high proportion of X-radiation has penetrated the anatomy to expose the radiographic element. The areas with high levels of barium often appear as physiological featureless white areas, since very little X-radiation is transmitted through the barium rich regions of the anatomy.

A well known procedure in the diagnosis and subsequent treatment of tumors involves marking a suspected tumor with radioactivity-tagged materials generally known as radiopharmaceuticals (such as ^{99M}technetium or ⁶⁷gallium, for example), which are administered orally or intravenously. The radiopharmaceuticals tend to concentrate in the area of a tumor, and the uptake of such radiopharmaceuticals in the active part of a tumor is higher and more rapid than in the tissue that neighbors the tumor.

Radiopharmaceuticals have been used in gastrointestinal studies. For example, U.S. Pat. Nos. 5,657,759 and 6,132,372 to Essen-Moller, discuss prior art methods of assessing gastric emptying with the use of radionuclide-labeled meals. In the prior art, gastric emptying of radiolabeled solids and liquids may be evalunted simultaneously when the various phases are marked with different tracers. Frequently used radiolabeled solid and liquid meals include chicken liver, eggs, oatmeal, orange juice and water. After ingestion of a labeled meal, anterior and posterior gamma camera images of the stomach area are obtained in 5 to 15 minute intervals for 1.5 to 2.0 hours After correction for decay, the counts in the gastric area are plotted as percentages of total counts at the start of imaging. Results are often presented as curves of emptying for liquids or solids against time, with the 5th and 95th percentiles of normals for comparison. Another technique of assessment, which is simpler to employ, is to derive the “emptying half-time” (not to be confused with radioactive decay), that is, the time taken to empty 50% of a meal from the stomach.

However, these prior art methods can only be performed with the patient immobilized, which necessitates using relatively short time periods (e.g., 2 hours) which do not adequately match the physiological environment associated with meals.

U.S. Pat. Nos. 5,657,759 and 6,132,372 describe a system and a method for gastric emptying and gastrointestinal output using an intragastrointestinal catheter. Specifically, the system includes an intragastrointestinal isotope activity sensor catheter, a combined stationary and ambulatory recorder, and a dedicated software program. The catheter includes a tubular body and one or more isotope activity sensors. Each isotope activity sensor communicates through the interior of the tubular body to the proximal end of the catheter and is used for the purpose of detecting isotope activity. The catheter is connected to an external preamplifier, which is connected to a multi-channel digital data acquisition recorder. The recorder records the number of counts per second as detected by the isotope activity sensors. Other parameters such as pH, pressure, EGG, IGG and bile may be recorded simultaneously. Measured data can be displayed in real time by means of an on-line interface on a computer screen or stored in the digital memory of the recorder.

However, such a catheter system causes logistic problems to the patient, limits the patient's freedom of movement and causes discomfort.

Another method known in the prior art for gastrointestinal examination of suspected tumors is magnetic resonance imaging (MRI). However, meaningful magnetic resonance images of the gastrointestinal tract require a suitable MRI contrast agent for the gastrointestinal tract. MRI contrast agents primarily act by affecting T1 or T2 relaxation of water protons. Contrast agents generally shorten T1 and/or T2. When contrast agents shorten T1, this increases signal intensity on T1 weighted images. When contrast agents shorten T2, this decreases signal intensity particularly on T2 weighted pulse sequences.

Several prototype gastrointestinal MRI contrast agents have been developed to assist abdominal MRI, but none have been completely satisfactory. For example, iron oxides, which are strong T2 relaxation agents, have been used as negative gastrointestinal MRI contrast agents to decrease signal intensity in the gastrointestinal tract. These agents, which predominantly affect T2 have the disadvantages of magnetic susceptibility artifacts, which occur as a result of detrimental effects on local magnetic homogeneity (magnetic susceptibility) caused by these agents. Magnetic susceptibility artifacts make it difficult to assess the bowel wall, bowel mesentery and adjacent structures.

The paramagnetic MRI contrast agent gadolinium-DTPA has also been tested as a positive gastrointestinal MRI contrast agent to increase signal intensity T1 weighted images, but this agent has the drawback that decomplexation and release of free gadolinium ion may occur in the gastrointestinal tract, which can be quite toxic. Furthermore, gadolinium-DTPA is relatively expensive.

Ferric iron has also been experimented with as an oral gastrointestinal MRI contrast agent. Ferric iron has been administered in the form of ferric ammonium citrate wherein the paramagnetic Fe⁺³ iron relaxes the water in the bowel to make the bowel bright on T1 weighted images. However, in order to obtain reasonable contrast enhancement, a relatively high dose of ferric iron is required, and some of this iron is absorbed as it passes down the gastrointestinal tract. Absorption of the iron creates two problems. First, absorption of the iron may cause problems with iron toxicity and iron overload. Second, as the iron is absorbed from the gastrointestinal tract, the concentration of the contrast agent decreases and the degree of contrast enhancement is much less in the distal bowel.

Manganese has been contemplated as a contrast agent for MRI. However, manganese, when administered intravenously as a contrast agent, may be teratogenic at clinical dosages. Administered intravenously, manganese is also known to interfere with the normal functioning of the heart by replacement of calcium in the calcium pump of the heart. In order to reduce the direct effect on the heart, oral administration of manganese has been proposed. A result of the vascularization of the upper gastrointestinal tract is that orally administered material taken up into the blood from the gut passes to the liver before passing to the heart. In the case of manganese, absorption by the hepatocytes in the liver prevents cardiotoxic levels of manganese reaching the heart. This hepatocyte uptake of manganese has led to the use of orally administered manganese as a liver imaging contrast agent.

Still another method is described in PCT International Application No. WO92/00402. This document describes a non-invasive method for detecting gastric epithelial damage using a disaccharide such as sucrose, maltose or lactose which is orally administered to a patient followed by assaying the patient's blood or urine for the disaccharide to determine the existence and extent of gastric epithelial damage. While this method overcomes the problems associated with invasive or radioisotopic methods, it does not reliably detect damage of the intestinal tract.

Yet another method the prior at for gastrointestinal examination of suspected tumors is the use of a small ingested pill that travels in the digestive tract. This pill contains a small video camera, a light source and position sensing. The pill is powered internally and sends the information to an external unit that is worn by the patient.

An article entitled, “Improved sensor pills for physiological monitoring,” by Lin et al., NASA Tech Brief, February 2000, 25(2), from JPL New Technology Report, NPO-20652, describes ingestible pills enabled to measure temperature and to sense the presence of blood, bacteria, and various chemicals in the gastrointestinal tract of a subject. Similarly, U.S. Pat. Nos. 5,279,607 and 5,395,366 describe ingestible pills which deliver or retrieve a substance from a designated site in the gastrointestinal tract.

U.S. Pat. No. 6,082,366 to Andra et al., describes apparatus for determining the position and speed of passage of a probe in the gastrointestinal tract.

Prior art ingestible pills suffer from a number of drawbacks. First, the digestive tract needs to be cleared. This causes discomfort to the patients. Second, the camera sees a large angle and hence, it is difficult to see small legions. Third, the pill travels relatively fast since the digestive tract is only filled with liquids, which means that some areas may be missed. Fourth, the viewing window of the camera may be blurred during the procedure, by feces and other intestinal substances. Fifth, the pill is very expensive and has to be retrieved at the end of the procedure for reuse, thus requiring recharging the batteries and re-sterilization.

There is thus a widely recognized need for, and it would be highly advantageous to have, a device and method for detecting pathologies in the gastrointestinal tract devoid of the above limitations.

### SUMMARY OF THE INVENTION

Thus, the present invention seeks to provide an improved method and system for tracking and communicating with a diagnostic instrument that travels in the gastrointestinal tract. The invention includes a sensor, preferably in the form of an ingestible pill, tablet, capsule, bolus and the like (the terms being used interchangeably herein). The present invention enables measuring the length that the sensor has traveled in the gastrointestinal tract from a reference point to a site of interest (such as an aberrant structure or growth in the gastrointestinal tract), no matter how convoluted the portion of the tract may be. Measurement of the length through the gastrointestinal tract is generally independent of wave-like motion of the gastrointestinal tract and of movement of the patient's body.

The sensor preferably includes a position detector, which outputs positional information about the position of the sensor in the gastrointestinal tract. In one embodiment, the position detector is internal to the sensor and transmits the positional information to an external position tracking system. Alternatively, the position detector may be external to the sensor and be part of the position tracking system.

The sensor also preferably includes a physiological detector, which outputs physiological information about a characteristic physiological feature of tissue in the gastrointestinal tract. Examples of characteristic physiological features of tissue in the gastrointestinal tract include absorptivity of radiopharmaceuticals, reflectivity of sound or light, smoothness or roughness of the intestinal wall and the like. The physiological detector preferably outputs the detected information to a physiological detector system.

A processor processes the information received by the position tracking system and the physiological detector system. The processor correlates the position of the sensor with the characteristic physiological feature of the tissue detected by the sensor at the position of the sensor.

For example, the physiological detector may comprise a plurality of radiation detectors, which detect radiation given off by structures within the gastrointestinal tract after administration of a radiopharmaceutical. The processor correlates the distance through the gastrointestinal tract with the aberrant structures found therein by radiation counts of the radiation detectors. This provides medical personnel with the vital data of the location and nature of the aberrant structure in the gastrointestinal tract.

The invention overcomes the drawbacks of the optically-based pill of the prior art, as described hereinabove, thereby enabling screening of large populations with relatively small discomfort for patients and high sensitivity even for small tumors.

There is thus provided in accordance with a preferred embodiment of the present invention gastrointestinal-tract apparatus including a sensor adapted to move along and measure a length of a gastrointestinal tract from a reference point to a site of interest in the gastrointestinal tract.

In accordance with a preferred embodiment of the present invention a position detector is adapted to output positional information about a position of the sensor in the gastrointestinal tract.

In accordance with one embodiment of the present invention, the position detector is internal to the sensor and is adapted to transmit the positional information to a position tracking system. In accordance with another embodiment of the present invention, the position detector is external to the sensor and is adapted to track the position of the sensor in the gastrointestinal tract, and transmit the positional information to a position tracking system.

Further in accordance with a preferred embodiment of the present invention the sensor includes a physiological detector adapted to output physiological information about a characteristic physiological feature of tissue in the gastrointestinal tract.

Still further in accordance with a preferred embodiment of the present invention a processor is adapted to correlate the position of the sensor with the characteristic physiological feature of the tissue detected by the sensor at the position of the sensor.

Additionally, in accordance with a preferred embodiment of the present invention, the sensor includes an ingestible pill.

In accordance with a preferred embodiment of the present invention the physiological detector includes a radiation detector responsive to a radiopharmaceutical administered to the gastrointestinal tract.

Further in accordance with a preferred embodiment of the present invention the radiation detector includes a gamma or beta radiation sensor.

In accordance with a preferred embodiment of the present invention the sensor includes a plurality of radiation detectors placed at different positions in the sensor

Further in accordance with a preferred embodiment of the present invention the processor calculates a distance traveled by the sensor as a function of radiation counts from the plurality of radiation detectors that are counted per unit time.

In accordance with another preferred embodiment of the present invention the physiological detector includes a plurality of ultrasound sensors placed at different positions in the sensor.

Further in accordance with a preferred embodiment of the present invention the processor calculates a distance traveled by the sensor as a function of an amplitude of an ultrasonic pulse echoing off an internal wall of the gastrointestinal tract.

In accordance with yet another preferred embodiment of the present invention the physiological detector includes a plurality of light sensors placed at different positions in the sensor.

Further in accordance with a preferred embodiment of the present invention the processor calculates a distance traveled by the sensor as a function of an amplitude of a light pulse reflecting off an internal wall of the gastrointestinal tract.

Still further in accordance with a preferred embodiment of the present invention a power source is adapted to power the sensor, and moves along with the sensor.

Still further in accordance with a preferred embodiment of the present invention the processor calculates a distance traveled by the sensor as a function of accelerations sensed by an internal inertial sensor. The inertial sensor senses accelerations in at least three degrees of freedom, such as with respect to a set of three mutually perpendicular coordinate axes.

In accordance with a preferred embodiment of the present invention an external magnetic navigation system is adapted to track a position of the sensor through the gastrointestinal tract.

Further in accordance with a preferred embodiment of the present invention the external magnetic navigation systems is adapted to sense the position of the sensor at a first position and at a second position in a coordinate system, and calculate the distance between the first and second positions.

Still further in accordance with a preferred embodiment of the present invention the external magnetic navigation system is adapted to sense the position of the sensor at predetermined time intervals.

In accordance with a preferred embodiment of the present invention the sensor includes a plurality of rotatable, at least partially round elements. The processor preferably calculates a distance traveled by the sensor as a function of time-based rotation of the at least partially round elements. The processor may be in optical or magnetic communication with the sensor, for example.

In accordance with a preferred embodiment of the present invention the sensor includes a memory device adapted to record data from the position detector or the physiological detector.

Further in accordance with a preferred embodiment of the present invention there is provided a reader adapted to access and read data stored in the memory device.

There is also provided in accordance with a preferred embodiment of the present invention a method for detecting a site of interest in a gastrointestinal tract, the method including sensing a length traveled by a sensor in a gastrointestinal tract from a reference point to a site of interest in the gastrointestinal tract.

The method preferably further includes collecting and/or transmitting positional information about a position of the sensor in the gastrointestinal tract, and sensing a characteristic physiological feature of tissue in the gastrointestinal tract.

Further in accordance with a preferred embodiment of the present invention the position of the sensor is correlated with the characteristic physiological feature of the tissue.

Implementation of the method and system of the present invention involves performing or completing selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### BRIEF DESCRIPTION OF THE DRAWINGS

The invention is herein described, by way of example only, with reference to the accompanying drawings. With specific reference now to the drawings in detail, it is stressed that the particulars shown are by way of example and purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a simplified pictorial illustration of gastrointestinal-tract apparatus, constructed and operative in accordance with a preferred embodiment of the present invention;

FIG. 2 is a simplified pictorial illustration of a sensor traveling through a gastrointestinal tract towards an aberrant structure in the gastrointestinal tract;

FIG. 3 is a simplified block diagram of the apparatus of FIG. 1, in accordance with a preferred embodiment of the present invention;

FIG. 4 is a simplified block diagram of a sensor with a radiation detector incorporated therein used in the apparatus of FIG. 1;

FIG. 5 is a simplified block diagram of the apparatus of FIG. 1 incorporated in a position tracking system, constructed and operative in accordance with a preferred embodiment of the present invention;

FIG. 6 is a simplified pictorial illustration of apparatus for tracking the position of the sensor of FIG. 2, in accordance with another embodiment of the present invention, wherein the sensor includes a plurality of rotatable, at least partially round elements;

FIG. 7 is a simplified pictorial illustration of apparatus for tracking the position of the sensor of FIG. 2, in accordance with yet another embodiment of the present invention, wherein the sensor includes an inertial sensor that senses accelerations in at least three degrees of freedom; and

FIG. 8 is a simplified pictorial illustration of gastrointestinal-tract apparatus, constructed and operative in accordance with another preferred embodiment of the present invention, wherein a sensor records data as it passes through the gastrointestinal tract.

### DETAILED DESCRIPTION OF A PREFERRED EMBODIMENT

The present invention is of a method and system for tracking and communicating with a diagnostic instrument that travels in the gastrointestinal tract which can be used to detect pathologies therein. The present invention can be used to detect tissues in the gastrointestinal tract that are afflicted as a result of gastrointestinal tumors, irritable bowel syndrome, fluxional diarrhea, ulcerative colitis, collagenous colitis, microscopic colitis, lymphocytic colitis, inflammatory bowel disease, Crohn's disease, infectious diarrhea, ulcerative bowel disease, lactase deficiency, infectious diarrhea, amebiasis, and giardiasis, for example.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

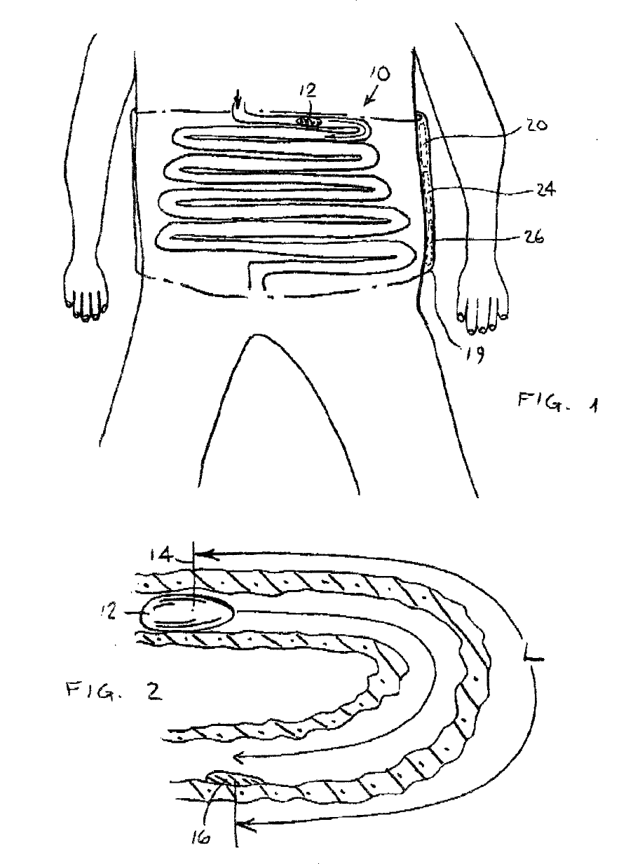

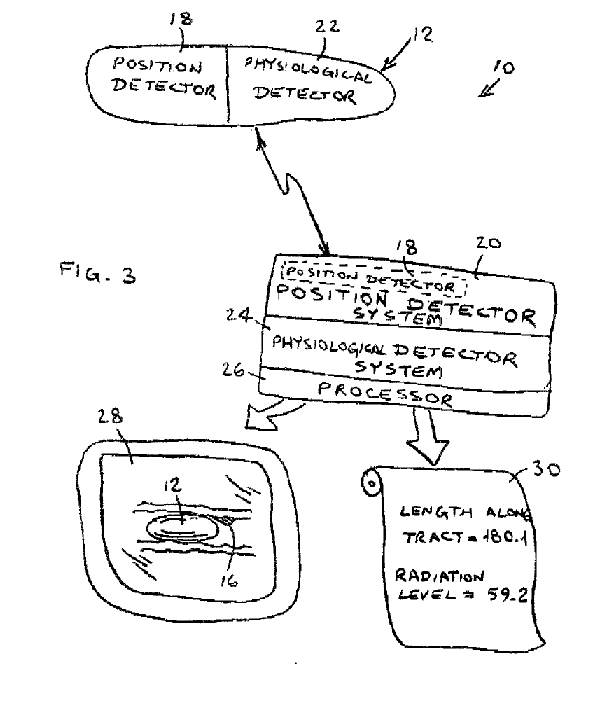

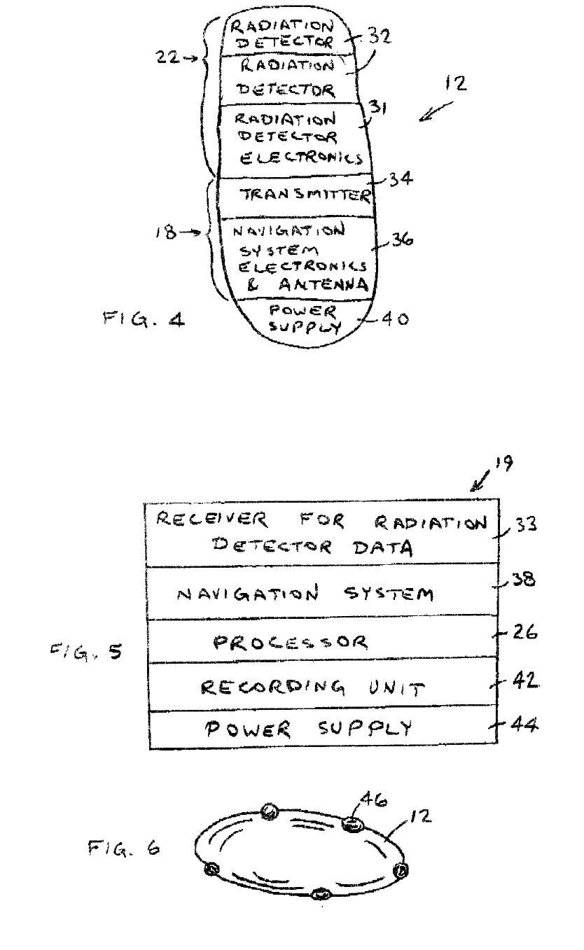

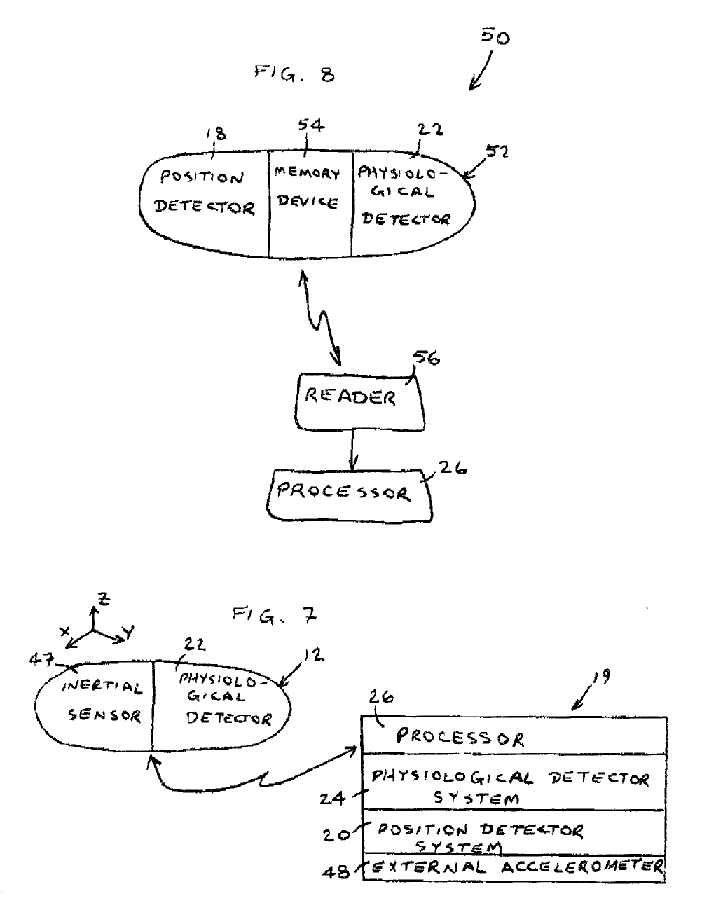

Reference is now made to FIGS. 1 and 2, which illustrate gastrointestinal-tract apparatus 10, constructed and operative in accordance with a preferred embodiment of the present invention.

Apparatus 10 includes a sensor 12, preferably in the form of an ingestible pill, tablet, capsule, bolus and the like. The present invention enables measuring the length L (FIG. 2) that sensor 12 has traveled in the gastrointestinal tract from a reference point 14 to a site of interest 16 (such as an aberrant structure or growth in the gastrointestinal tract), no matter how convoluted the portion of the tract may be. Measurement of length L is generally independent of wave-like motion of the gastrointestinal tract and of movement of the patient's body, as explained hereinbelow.

Reference is now made to FIG. 3, which illustrates sensor 12 in greater detail. Sensor 12 preferably includes a position detector 18 adapted to output positional information about a position of sensor 12 in the gastrointestinal tract. In one embodiment, position detector 18 is internal to sensor 12 and either stores positional information in a memory device located in sensor 12 and/or transmits the positional information to an external position tracking system 20. Examples of such an embodiment are described in greater detail hereinbelow. Alternatively, position detector 18 may be external to sensor 12 and be part of position tracking system 20. An example of such an embodiment is described in greater detail hereinbelow.

Sensor 12 also preferably includes a physiological detector 22 adapted to output physiological information about a characteristic physiological feature of tissue in the gastrointestinal tract. Examples of characteristic physiological features of tissue in the gastrointestinal tract include absorptivity of radiopharmaceuticals, reflectivity of sound or light, smoothness or roughness of the intestinal wall and the like. Physiological detector 22 preferably outputs the detected information to a physiological detector system 24.

A processor 26 is provided that processes the information received by position tracking system 20 and physiological detector system 24, and correlates the position of sensor 12 with the characteristic physiological feature of the tissue detected by sensor 12 at the position of sensor 12. Processor 26 may be in wireless communication with sensor 12 for controlled operation in a closed control loop. As seen in FIG. 1, position tracking system 20, physiological detector system 24 and processor 26 may be incorporated in an external device 19, such as a belt or other similar device worn by the patient.

Processor 26 preferably outputs the position of sensor 12 and the detected physiological feature of the tissue, such as visually to a monitor 28 and/or a chart recorder 30, which spells out the detected data, such as length traveled along the tract and radiation level at a certain point.

Reference is now made to FIGS. 4 and 5, which illustrate a preferred embodiment of the invention wherein physiological detector 22 comprises a radiation detector 32 responsive to a radiopharmaceutical administered to the gastrointestinal tract Radiation detector 32 may be, for example, a gamma or beta radiation sensor, and is preferably omni-directional, i.e., receives radiation from 360°. Moreover, there may be a plurality of radiation detectors 32 placed at different positions in sensor 12. Radiation detector electronics 31 are preferably provided for outputting and transmitting data to a receiver 33 for the radiation detector data.

In carrying out the invention with this embodiment, a patient to be screened is first injected with a small amount of radiopharmaceutical (such as monoclonal antibodies or other agents, e.g., fibrinogen or fluorodeoxyglucose tagged with a radioactive isotope, e.g., ^{99M}technetium, ⁶⁷gallium, ²⁰¹thallium, ¹¹¹indium, ¹²³iodine, ¹²⁵iodine and ¹⁸fluorine). Alternatively, the radiopharmaceutical may be administered orally.

Position detector 18 of sensor 12 preferably includes a transmitter 34 and a navigation system electronics and antenna unit 36 for communicating positional information to a navigation system 38, and for receiving feedback from the navigation system 38. Examples of systems that include such a transmitter 34, navigation system electronics and antenna unit 36, and navigation system 38 are the magnetic tracking and location systems commercially available from Ascension Technology Corporation, P.O. Box 527, Burlington, Vt. 05402 USA (http://www.ascension-tech.com/graphic.htm). The magnetic tracking and location systems of Ascension Technology Corporation use DC magnetic fields to overcome blocking and distortion from nearby conductive metals. Signals pass through the human body without attenuation.

A power supply 40 is preferably contained within sensor 12 for powering the various components of position detector 18 and physiological detector 22. The components may be alternatively powered externally through induction coils. Receiver 33 and navigation system 38 are preferably in communication with processor 26 in external device 19. A recording unit 42 may be provided for recording the processed data. A power supply 44 is preferably provided for powering the various units of external device 19.

After administration of the radiopharmaceutical, tumors or inflammations in the digestive tract emit radiation with higher density compared with their surroundings. Sensor 12 is then ingested by the patient, and its position is tracked by navigation system 38. Radiation detector 32 measures the radioactivity of structures within the gastrointestinal tract as sensor 12 travels therethrough. Preferably an algorithm and timer in processor 26 compensates for time with regards to the radiation counts, as the radiopharmaceutical agent naturally decays. Processor 26 correlates distance through the gastrointestinal tract with the aberrant structures found therein by the radiation counts.

There are many medical applications for which such use of radiopharmaceutical agents is particularly advantageous. For example, one important application of sensor 12 with radiation detectors 32 is in the detection of small intestine bleeding locations such as in Crohn's disease. Crohn's disease is characterized, inter alia, by recurrent breakages of the intestine blood vessels and bleeding therein, which becomes manifested by the feces being black. However, the actual position of blood leakage is generally difficult, if not impossible, to pinpoint in the prior art. Typically, in the prior art, treatment involves surgically removing the affected portion of the intestine. Without positional knowledge, the surgeon has difficulty searching for the bleeding source and typically, larger chunks of intestine must be removed. In contrast, in the present invention, after introducing a blood-administered radiopharmaceutical, sensor 12 with radiation detectors 32 can provide positional data to pinpoint the source of bleeding.

There are different methods of sensing the distance traveled by sensor 12 through the gastrointestinal tract. For example, navigation system 38 may be an external magnetic navigation system (such as the magnetic tracking and location systems of Ascension Technology Corporation). Such a system tracks the location of sensor 12 by assigning new reference coordinates to the position of sensor 12 every few seconds and calculating the distance traveled between each position. In this way, movements of the gastrointestinal tract have negligible effects on the overall accuracy of the calculation of the length traveled within the gastrointestinal tract. This is an example of position detector 18 being external to sensor 12 and part of position tracking system 20.

In other embodiments, as mentioned hereinabove, position detector 18 is internal to sensor 12 and transmits the positional information to position tracking system 20 By using an internal position detector 18, sensor 12 measures and calculates the actual distance traveled within the gastrointestinal tract, and transmits the distance at short intervals of time to external position tracking system 20. Examples of internal position detectors include ultrasonic detectors, gamma radiation detectors, beta radiation detectors, light emitting diodes and light detectors. For example, if gamma or beta radiation detectors are used for the internal navigation, the detectors are placed in different parts of sensor 12. The radiation counts from the various detectors are counted per unit time. Processor 26 calculates the distance traveled by sensor 12 as a function of radiation counts from the plurality of radiation detectors by cross correlating the counts from the different detectors at a first time T and at a second time T−t. The distance traveled is proportional to the cross correlation. This type of relative movement tracking is well known in the art and is utilized in a number of products such as the Logitec iFeel™ MouseMan.

If ultrasound is used, a plurality of transmitters and receivers are placed at different locations in sensor 12. The transmitters transmit ultrasound pulses, which echo off the gastrointestinal tract walls. The receivers receive the bounced echoes, which have different amplitudes of received signals. Processor 26 calculates the distance traveled by sensor 12 as a function of the amplitude of the ultrasonic pulse echoing off the internal wall of the gastrointestinal tract, by cross correlating the signals from the different detectors at a first time T and at a second time T+t. The distance traveled is proportional to the cross correlation. This type of relative movement tracking is well known in the art and is utilized in a number of products such as the Logitec iFeel™ MouseMan.

If light is used for internal position tracking, small light emitting diodes are placed at different locations in sensor 12 and small light sensitive detectors detect the light bounced off the gastrointestinal tract walls As with the ultrasound, the receiving sensors have different amplitudes of received signals. Processor 26 calculates the distance traveled by sensor 12 as a function of the amplitude of the light pulse reflecting off the internal wall of the gastrointestinal tract, by cross correlating the signals from the different detectors at a first time T and at a second time T+t. The distance traveled is proportional to the cross correlation. This type of relative movement tracking is well known in the art and is utilized in a number of products such as the Logitec iFeel™ MouseMan.

Reference is now made to FIG. 6, which illustrates yet another way of tracking sensor 12, in accordance with another embodiment of the present invention. In this embodiment, sensor 12 includes a plurality of rotatable, at least partially round elements 46, such as spherical or cylindrical rollers, attached to the surface of sensor 12. Processor 26 calculates the distance traveled by sensor 12 as a function of the time-based rotation of elements 46. Processor 26 may be in optical or magnetic communication with sensor 12, for example, for sensing the rotation of elements 46.

Reference is now made to FIG. 7, which illustrates still another way of tracking sensor 12, in accordance with another embodiment of the present invention. In this embodiment, the position detector of sensor 12 includes an internal inertial sensor 47 which senses accelerations in at least three degrees of freedom, such as with respect to a set of three mutually perpendicular coordinate axes, inertial sensor 47 may comprise one or more miniature or micro-accelerometers, which sense accelerations in at least three degrees of freedom, such as with respect to a set of three mutually perpendicular coordinate axes, X, Y and Z. Processor 26 may be in wireless communication with inertial sensor 47, such as by a BLUETOOTH wireless connection, for example. Processor 26 calculates a distance traveled by sensor 12 as a function of the accelerations sensed by inertial sensor 47, the accelerations being related to distance traveled, as is well known in the art. Processor 26 sums the distances directed along the length of the gastrointestinal tract to arrive at the distance that sensor 12 has traveled in the gastrointestinal tract.

In the embodiment of FIG. 7, external device 19 preferably includes an external accelerometer 48, which is adapted to sense accelerations of the patient's body. Accelerometer 48 enables processor 26 to take into account the differences between the accelerations of sensor 12 and the accelerations of external device 19 worn by the patient. Any acceleration that is common to both is related to movements of the patient and not to movements of sensor 12 with respect to the patient's body. Thus, processor 26 does not take into account such common accelerations when calculating the distance traveled by sensor 12 along the gastrointestinal tract.

Reference is now made to FIG. 8, which illustrates gastrointestinal-tract apparatus 50, constructed and operative in accordance with another preferred embodiment of the present invention. Apparatus 50 includes a sensor 52 which is a modification of sensor 12. Sensor 52 comprises a memory device 54 for recording the positional data from position detector 18 and physiological data from physiological detector 22. Memory device 54 may be any kind of memory device in which information may be stored and retrieved therefrom, such as, but not limited to, non-volatile memory arrays, for example, flash memory arrays or erasable, programmable read only memory (EPROM) arrays.

In one preferred embodiment, the positional and physiological data is collected by sensor 52 and stored therein for reading upon exiting the body from the rectum. A reader 56 is provided for accessing, retrieving and reading the stored data. Reader 56 may be in wired or wireless communication with memory device 54 of sensor 52, such as by a BLUETOOTH wireless connection, for example. Processor 26 then processes the information as described hereinabove.

Different variations of apparatus 50 are also possible. For example, time-related positional data may be communicated or collected outside the body in real time, whereas physiological data may be recorded in memory device 54 in a time-dependent manner to be retrieved when sensor 52 exits the body. Conversely, physiological data may be communicated or collected outside the body in real time, whereas positional data may be recorded in memory device 54 in a time-dependent manner to be retrieved when sensor 52 exits the body.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art (e.g., the sensor described herein may be used to detect pathologies outside of the gastrointestinal tract, such as prostate cancer). Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. Gastrointestinal-tract apparatus comprising:
a sensor adapted to move along and measure a length of a gastrointestinal tract from a reference point to a site of interest in the gastrointestinal tract.

2. Apparatus according to claim 1 wherein said sensor comprises a physiological detector adapted to output physiological information about a characteristic physiological feature of tissue in the gastrointestinal tract.

3. Apparatus according to claim 1 and comprising an ingestible pill, wherein said sensor is substantially internal to said pill.

4. Apparatus according to claim 1 and comprising a position detector adapted to output positional information about a position of said sensor in the gastrointestinal tract.

5. Apparatus according to claim 4 wherein said position detector is internal to said sensor and is adapted to transmit the positional information to a position tracking system.

6. Apparatus according to claim 4 wherein said position detector is external to said sensor and is adapted to track the position of said sensor in the gastrointestinal tract, and transmit the positional information to a position tracking system.

7. Apparatus according to claim 4 wherein said sensor comprises a memory device adapted to record data from said position detector.

8. Apparatus according to claim 7 and comprising a reader adapted to read data stored in said memory device.

9. Apparatus according to claim 4 wherein said sensor comprises a physiological detector adapted to output physiological information about a characteristic physiological feature of tissue in the gastrointestinal tract.

10. Apparatus according to claim 9 wherein said sensor comprises a memory device adapted to record data from said physiological detector.

11. Apparatus according to claim 10 and comprising a reader adapted to read data stored in said memory device.

12. Apparatus according to claim 9 and comprising a processor adapted to correlate the position of said sensor with the characteristic physiological feature of the tissue detected by said sensor at the position of said sensor.

13. Apparatus according to claim 9 wherein said physiological detector comprises a radiation detector adapted to output the physiological information responsive to a level of radiation generated by a radiopharmaceutical.

14. Apparatus according to claim 13 wherein said radiation detector comprises a gamma radiation sensor.

15. Apparatus according to claim 13 wherein said radiation detector comprises a beta radiation sensor.

16. Apparatus according to claim 13 wherein said physiological detector comprises a plurality of radiation detectors placed at different positions in said sensor.

17. Apparatus according to claim 16 and comprising a processor in communication with said sensor, wherein said processor is adapted to calculate a distance traveled by said sensor as a function of radiation counts per unit time generated by said plurality of detectors.

18. Apparatus according to claim 9 wherein said physiological detector comprises a plurality of ultrasound sensors placed at different positions in said sensor.

19. Apparatus according to claim 18 and comprising a processor in communication with said sensor, wherein said processor is adapted to calculate a distance traveled by said sensor as a function of an amplitude of an ultrasonic pulse echoing off an internal wall of the gastrointestinal tract.

20. Apparatus according to claim 9 wherein said physiological detector comprises a plurality of light sensors placed at different positions in said sensor.

21. Apparatus according to claim 20 and comprising a processor in communication with said sensor, wherein said processor is adapted to calculate a distance traveled by said sensor as a function of an amplitude of a light pulse reflecting off an internal wall of the gastrointestinal tract

22. Apparatus according to claim 9 wherein said physiological detector comprises a plurality of physiological detectors placed at different positions in said sensor, wherein the apparatus comprises a processor in communication with said sensor, and wherein said processor is adapted to calculate a distance traveled by said sensor responsive to signals generated by said plurality of physiological detectors.

23. Apparatus according to claim 1 and comprising a power source adapted to power said sensor, wherein said power source is adapted to move along with said sensor.

24. Apparatus according to claim 1 and comprising an external magnetic navigation system adapted to track a position of said sensor through the gastrointestinal tract.

25. Apparatus according to claim 24 wherein said external magnetic navigation system is adapted to sense the position of said sensor at a first position and at a second position in a coordinate system, and calculate the distance between the first and second positions.

26. Apparatus according to claim 24 wherein said external magnetic navigation system is adapted to sense the position of said sensor at predetermined time intervals.

27. Apparatus according to claim 1 wherein said sensor comprises a plurality of rotatable, at least partially round elements disposed on an outer surface of the sensor.

28. Apparatus according to claim 27 and comprising a processor in communication with said sensor, wherein said processor calculates a distance traveled by said sensor as a function of rotation of said at least partially round elements.

29. Apparatus according to claim 28 wherein said processor is in optical communication with said sensor.

30. Apparatus according to claim 28 wherein said processor is in magnetic communication with said sensor.

31. Apparatus according to claim 4 wherein said position detector comprises an inertial sensor that senses accelerations of said sensor in at least three degrees of freedom.

32. Apparatus according to claim 31 and comprising a processor adapted to correlate the position of said sensor with the accelerations sensed by said inertial sensor.

33. Apparatus according to claim 32 and comprising an external accelerometer adapted to be worn by a patient and to sense accelerations of the patient's body.

34. Apparatus according to claim 33 wherein said processor is adapted to take into account differences between accelerations sensed by said inertial sensor and accelerations sensed by said external accelerometer.

35. A method for sensing in a gastrointestinal tract, the method comprising:
sensing a length traveled by a sensor in a gastrointestinal tract from a reference point to a site of interest in the gastrointestinal tract.

36. The method according to claim 35 wherein sensing the length comprises sensing the length traveled by a sensor included in an ingestible pill.

37. The method according to claim 35 and comprising sensing positional information about a position of said sensor in the gastrointestinal tract.

38. The method according to claim 37 and comprising sensing a characteristic physiological feature of tissue in the gastrointestinal tract.

39. The method according to claim 38 and comprising correlating the position of said sensor with the characteristic physiological feature of the tissue.

40. The method according to claim 38 wherein sensing said characteristic physiological feature comprises sensing a radiation level due to a radiopharmaceutical.

41. The method according to claim 35 wherein sensing the length comprises calculating a distance traveled by said sensor as a function of radiation counts per unit time generated by a plurality of radiation detectors coupled to said sensor.

42. The method according to claim 35 wherein sensing the length comprises:
detecting, with a plurality of ultrasound sensors coupled to the sensor, an ultrasonic pulse echoing off an internal wall of the gastrointestinal tract; and
calculating a distance traveled by said sensor as a function of an amplitude of the ultrasonic pulse.

43. The method according to claim 35 wherein sensing the length comprises:
detecting, with a plurality of light sensors coupled to the sensor, a light pulse reflecting off an internal wall of the gastrointestinal tract; and
calculating a distance traveled by said sensor as a function of an amplitude of the light pulse.

44. The method according to claim 35 wherein sensing the length comprises:
detecting accelerations of said sensor as said sensor moves in the gastrointestinal tract; and
calculating a distance traveled by said sensor as a function of said accelerations.

45. The method according to claim 44 wherein the gastrointestinal tract is in a patient's body, and the method further comprises detecting accelerations of the patient's body, wherein said calculating does take not into account any acceleration that is common to both said inertial sensor and the patient's body.

46. The method according to claim 35 and comprising recording in said sensor positional information about a position of said sensor in the gastrointestinal tract.

47. The method according to claim 46 and comprising reading said positional information after said sensor passes from the gastrointestinal tract.

48. The method according to claim 35 and comprising recording in said sensor information about a characteristic physiological feature of tissue in the gastrointestinal tract.

49. The method according to claim 48 and comprising reading said information about a characteristic physiological feature of tissue, after said sensor passes from the gastrointestinal tract.

50. An ingestible pill, comprising:
a radiation detector adapted to measure, at a plurality of sites of a gastrointestinal tract, radiation generated by a radiopharmaceutical.

51. The pill according to claim 50, wherein said radiation detector comprises a gamma radiation sensor.

52. The pill according to claim 50 wherein said radiation detector comprises a beta radiation sensor.

53. The pill according to claim 50 and comprising a position detector, adapted to output positional information about a position of said pill in the gastrointestinal tract.

54. The pill according to claim 53, wherein said position detector comprises at least one ultrasound detector, fixed to the pill.

55. The pill according to claim 53, wherein said position detector comprises at least one light detector, fixed to the pill.

56. The pill according to claim 53, wherein said position detector comprises a plurality of radiation detectors, disposed at respective sites of the pill.

57. The pill according to claim 50, wherein said radiation detector is adapted to measure radiation generated by a radiopharmaceutical administered to the gastrointestinal tract.

58. The pill according to claim 50, wherein said radiation detector is adapted to measure radiation generated by a radiopharmaceutical administered to a site external to the gastrointestinal tract.

59. The pill according to claim 58, wherein said radiation detector is adapted to measure radiation generated by a radiopharmaceutical administered intravenously.

60. A method for measuring radiation, comprising:
administering an ingestible pill including a radiation detector adapted to measure, at a plurality of sites of a gastrointestinal tract, radiation generated by a radiopharmaceutical.
